(19) United States
(12) Patent Application Publication
(10) Pub. No.: US 2023/0135286 A1
(43) Pub. Date: May 4, 2023

(54) SYSTEMS, DEVICES, AND METHODS FOR TRACKING ONE OR MORE SURGICAL LANDMARKS

(71) Applicant: Medtronic Navigation, Inc., Louisville, CO (US)

(72) Inventor: Saba Pasha, Houston, TX (US)

(21) Appl. No.: 17/516,246

(22) Filed: Nov. 1, 2021

(51) Int. Cl. A61B 34/20; A61B 34/32; G06T 7/00

(52) U.S. Cl. CPC A61B 34/20; A61B 34/32; G06T 7/0014; A61B 2034/2068; A61B 2034/105

(57) ABSTRACT

Systems, methods and devices for tracking one or more surgical landmarks are provided. A first image depicting one or more surgical landmarks may be received from an imaging device and a second image depicting the one or more surgical landmarks may be received after the first image. Movement of at least one of the one or more surgical landmarks may be detected and a notification for an operating room personnel may be generated when the detected movement meets or exceeds a movement threshold.

### BACKGROUND

The present disclosure is generally directed to tracking, and relates more particularly to tracking one or more surgical landmarks.

Surgical robots may assist a surgeon or other medical provider in carrying out a surgical procedure, or may complete one or more surgical procedures autonomously. Imaging may be used by a medical provider for diagnostic and/or therapeutic purposes. Patient anatomy can change over time, particularly following placement of a medical implant in the patient anatomy.

### BRIEF SUMMARY

Example aspects of the present disclosure include:

A system for tracking one or more surgical landmarks according to at least one embodiment of the present disclosure comprises an imaging device; a processor; and a memory storing data for processing by the processor, the data, when processed, causing the processor to: receive a first image depicting one or more surgical landmarks; receive a second image from the imaging device depicting the one or more surgical landmarks, the second image received after the first image; detect movement of at least one surgical landmark of the one or more surgical landmarks based on a comparison of the first image with the second image; and generate a notification for an operating room personnel when the detected movement meets or exceeds a movement threshold.

Any of the aspects herein, wherein the imaging device obtains images of a patient, including the second image, free of ionizing radiation.

Any of the aspects herein, wherein the one or more surgical landmarks comprise a reference marker disposed on corresponding anatomical elements, one or more implants implanted on corresponding anatomical elements, one or more anatomical elements, and/or a portion of each of one or more anatomical elements.

Any of the aspects herein, wherein detecting the movement of the at least one surgical landmark comprises comparing a position of the at least one surgical landmark in the first image to the position of the at least one surgical landmark in the second image.

Any of the aspects herein, wherein the first image is obtained preoperatively and the second image is obtained intraoperatively.

Any of the aspects herein, wherein the imaging device is a second imaging device and the system further comprises a first imaging device, wherein the first image is obtained from the first imaging device and the second image is obtained from the second imaging device.

Any of the aspects herein, wherein the first imaging device uses a first imaging modality and the second imaging device uses a second imaging modality different from the first imaging modality.

Any of the aspects herein, wherein the first image comprises a three-dimensional representation of the one or more surgical landmarks, wherein the first imaging device obtains images using ionizing radiation.

Any of the aspects herein, wherein the memory stores further data for processing by the processor that, when processed, causes the processor to: update at least a portion of the three-dimensional representation based on the second image.

Any of the aspects herein, wherein the second image comprises a two-dimensional representation of the one or more surgical landmarks.

Any of the aspects herein, wherein the memory stores further data for processing by the processor that, when processed, causes the processor to: update the three-dimensional representation of the at least one surgical landmark based on the detected movement of the at least one surgical landmark.

Any of the aspects herein, wherein the threshold is based on a predicted position of the at least one surgical landmark after a surgical step is performed.

Any of the aspects herein, wherein the second image is received in real-time.

A device for tracking an anatomical element according to at least one embodiment of the present disclosure comprises a processor; and a memory storing data for processing by the processor, the data, when processed, causing the processor to: receive a first image depicting one or more surgical landmarks; receive a second image depicting the one or more surgical landmarks, the second image received after the first image; detect movement of at least one surgical landmark of the one or more surgical landmarks based on the first image and the second image; and update the first image based on the detected movement.

Any of the aspects herein, wherein detecting the movement of the at least one surgical landmark comprises comparing a position of the at least one surgical landmark in the first image with a position of the at least one surgical landmark in the second image.

Any of the aspects herein, wherein the one or more surgical landmarks comprise a reference marker disposed on corresponding anatomical elements, one or more implants implanted on corresponding anatomical elements, one or more anatomical elements, and/or a portion of each of one or more anatomical elements.

Any of the aspects herein, wherein the first image is at least one of a two-dimensional or three-dimensional representation of the one or more surgical landmarks and the second image is at least one of a two-dimensional or three-dimensional representation of the one or more surgical landmarks, wherein the first image is obtained from a first imaging device using a first imaging modality and the second image is obtained from a second imaging device using a second imaging modality.

Any of the aspects herein, wherein the first imaging modality uses ionizing radiation and the second imaging modality is free of ionizing radiation.

Any of the aspects herein, wherein updating the first image occurs when the detected movement meets or exceeds a movement threshold.

A system for tracking one or more surgical landmarks according to at least one embodiment of the present disclosure comprises a first imaging device using a first imaging modality; a second imaging device using a second imaging modality; a processor; a memory storing data for processing by the processor, the data, when processed, causing the processor to: receive a first image from the first imaging device, the first image depicting one or more surgical landmarks; receive a second image from the second imaging device, the second image depicting the one or more surgical landmarks; detect movement of at least one surgical landmark of the one or more surgical landmarks based on the first image and the second image; and generate a notification when the detected movement meets a threshold.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a block diagram of a system according to at least one embodiment of the present disclosure;

FIG. 2 is a schematic illustration of one or more surgical landmarks according to at least one embodiment of the present disclosure; and

FIG. 3 is a flowchart according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia GeForce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

During a surgical operation (e.g., a spinal procedure), a plurality of short fluoroscopic images of anatomical elements (e.g., a spine) may be registered to a patient to provide a surgeon with feedback after a surgical step such as, for example, a screw placement has been performed. As such, surgeons do not receive any real-time feedback during the surgical operation. For example, in a spinal procedure, the surgeon may not receive feedback regarding a global alignment of the spine and whether the imparted changes during the surgery match the planned surgical outcome in terms of global spinal alignment.

According to at least one embodiment of the present disclosure methods, systems, and devices are provided to match intraoperative images of anatomical elements (e.g., a spine), after a surgical procedure (e.g., a screw placement), to a room level video, or room level images, of the anatomical elements. Anatomical landmarks such as screws, for example may be detected in the video or images. Recording the same landmarks during the surgical operation, using the videos or plurality of still images, allows creating global images of the anatomical elements (e.g., the spine) as various surgical moves are completed. This technology informs the surgeon of gradual changes to the anatomical elements and whether the optimal correction is achieved according to a preoperative surgical plan.

According to other embodiments of the present disclosure methods, systems, and devices are provided that use video/image analysis of spinal images during a spinal surgery to update an intraoperative spinal alignment image or three-dimensional representation. Screw trajectories may be determined via navigation, for example, and screw heads may be captured intraoperatively via video or a plurality of images. A preoperative image or an image taken prior to performing any correction, such as an initial long film of the spine, can be used as a reference image and changed gradually as the surgical procedure is carried out. Any tracking tool, which can be attached to the screws or bony landmarks, that provide correspondence between the initial intraoperatively image of the spine and movement of the screws or bony landmarks during the surgery, can provide real-time feedback to a surgeon, providing a more efficient execution of the surgical procedure. Combined with processed three-dimensional representations of the spine, such methods, systems, or devices can provide real-time information about a three-dimensional spinal alignment intraoperatively.

Embodiments of the present disclosure provide technical solutions to one or more of the problems of (1) tracking one or more surgical landmarks, (2) determining movement of one or more surgical landmarks, and/or (3) providing real-time positional information of one or more landmarks to a surgical team.

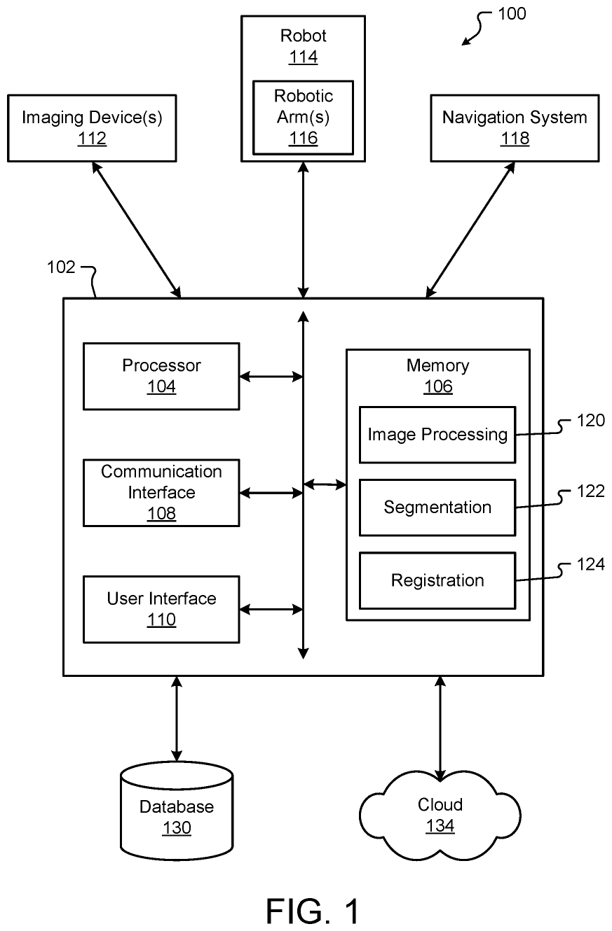

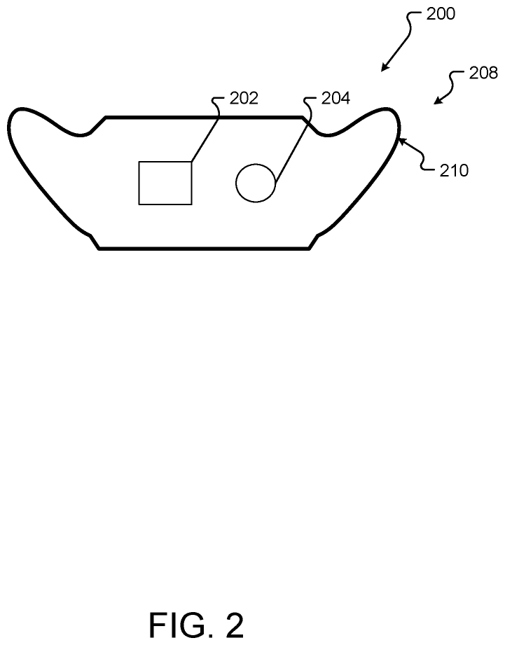

Turning first to FIG. 1, a block diagram of a system 100 according to at least one embodiment of the present disclosure is shown. The system 100 may be used to track one or more surgical landmarks (such as, for example, the one or more surgical landmarks 200 illustrated in FIG. 2) and/or carry out one or more other aspects of one or more of the methods disclosed herein. The system 100 comprises a computing device 102, one or more imaging devices 112, a robot 114, a navigation system 118, a database 130, and/or a cloud or other network 134. Systems according to other embodiments of the present disclosure may comprise more or fewer components than the system 100. For example, the system 100 may not include the imaging device 112, the robot 114, the navigation system 118, one or more components of the computing device 102, the database 130, and/or the cloud 134.

The computing device 102 comprises a processor 104, a memory 106, a communication interface 108, and a user interface 110. Computing devices according to other embodiments of the present disclosure may comprise more or fewer components than the computing device 102.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from the imaging device 112, the robot 114, the navigation system 118, the database 130, and/or the cloud 134.

The memory 106 may be or comprise RAM, DRAM, SDRAM, other solid-state memory, any memory described herein, or any other tangible, non-transitory memory for storing computer-readable data and/or instructions. The memory 106 may store information or data useful for completing, for example, any step of the method 300 described herein, or of any other methods. The memory 106 may store, for example, instructions and/or machine learning models that support one or more functions of the robot 114. For instance, the memory 106 may store content (e.g., instructions and/or machine learning models) that, when executed by the processor 104, enable image processing 120, segmentation 122, and/or registration 124. Such content, if provided as in instruction, may, in some embodiments, be organized into one or more applications, modules, packages, layers, or engines. Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the imaging device 112, the robot 114, the database 130, and/or the cloud 134.

The computing device 102 may also comprise a communication interface 108. The communication interface 108 may be used for receiving image data or other information from an external source (such as the imaging device 112, the robot 114, the navigation system 118, the database 130, the cloud 134, and/or any other system or component not part of the system 100), and/or for transmitting instructions, images, or other information to an external system or device (e.g., another computing device 102, the imaging device 112, the robot 114, the navigation system 118, the database 130, the cloud 134, and/or any other system or component not part of the system 100). The communication interface 108 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 108 may be useful for enabling the device 102 to communicate with one or more other processors 104 or computing devices 102, whether to reduce the time needed to accomplish a computing-intensive task or for any other reason.

The computing device 102 may also comprise one or more user interfaces 110. The user interface 110 may be or comprise a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some embodiments, the user interface 110 may be useful to allow a surgeon or other user to modify instructions to be executed by the processor 104 according to one or more embodiments of the present disclosure, and/or to modify or adjust a setting of other information displayed on the user interface 110 or corresponding thereto.

Although the user interface 110 is shown as part of the computing device 102, in some embodiments, the computing device 102 may utilize a user interface 110 that is housed separately from one or more remaining components of the computing device 102. In some embodiments, the user interface 110 may be located proximate one or more other components of the computing device 102, while in other embodiments, the user interface 110 may be located remotely from one or more other components of the computer device 102.

The imaging device 112 may be operable to image one or more landmarks (such as, for example, the one or more landmarks 200 depicted in FIG. 2). The one or more landmarks 200, as will be described in detail below, may comprise an anatomical element, a reference marker, an implant, or any object or component of a surgical setting (e.g., a surgical site, a surgical operating room, objects in a surgical operating room, people in a surgical operating room, etc.). As such, the imaging device may be operable to image anatomical feature(s) (e.g., a bone, veins, tissue, etc.), other aspects of patient anatomy, and/or any component of a surgical setting to yield image data (e.g., image data depicting or corresponding to a bone, veins, tissue, etc.). “Image data” as used herein refers to the data generated or captured by an imaging device 112, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure.

The imaging device 112 may be capable of taking/capturing a two-dimensional image or generate a three-dimensional representation to yield the image data. The imaging device 112 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MRI) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 112 suitable for obtaining images of an anatomical feature of a patient. The imaging device 112 may be contained entirely within a single housing, or may comprise a transmitter/emitter and a receiver/detector that are in separate housings or are otherwise physically separated.

In some embodiments, the imaging device 112 may comprise more than one imaging device 112. For example, a first imaging device may provide first image data and/or a first image, and a second imaging device may provide second image data and/or a second image. In still other embodiments, the same imaging device may be used to provide both the first image data and the second image data, and/or any other image data described herein. The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

In embodiments where the imaging device 112 comprising more than one imaging device, a first imaging device 112 may be used to obtain first image data (e.g., a first image) at a first time, and a second imaging device 112 may be used to obtain second image data (e.g., a second image) at a second time after the first time. In such embodiments, the first imaging device 112 may obtain first image(s) using a first imaging modality and the second imaging device 112 may obtain second image(s) using a second imaging modality. For example, the first imaging modality may use ionizing radiation (e.g., X-rays) and the second imaging modality may be free of ionizing radiation (e.g., ultrasound).

The robot 114 may be any surgical robot or surgical robotic system. The robot 114 may be or comprise, for example, the Mazor X™ Stealth Edition robotic guidance system. The robot 114 may be configured to position the imaging device 112 at one or more precise position(s) and orientation(s), and/or to return the imaging device 112 to the same position(s) and orientation(s) at a later point in time. The robot 114 may additionally or alternatively be configured to manipulate a surgical tool (whether based on guidance from the navigation system 118 or not) to accomplish or to assist with a surgical task. In some embodiments, the robot 114 may be configured to hold and/or manipulate an anatomical element during or in connection with a surgical procedure. The robot 114 may comprise one or more robotic arms 116. In some embodiments, the robotic arm 116 may comprise a first robotic arm and a second robotic arm, though the robot 114 may comprise more than two robotic arms. In some embodiments, one or more of the robotic arms 116 may be used to hold and/or maneuver the imaging device 112. In embodiments where the imaging device 112 comprises two or more physically separate components (e.g., a transmitter and receiver), one robotic arm 116 may hold one such component, and another robotic arm 116 may hold another such component. Each robotic arm 116 may be positionable independently of the other robotic arm. The robotic arms 116 may be controlled in a single, shared coordinate space, or in separate coordinate spaces.

The robot 114, together with the robotic arm 116, may have, for example, one, two, three, four, five, six, seven, or more degrees of freedom. Further, the robotic arm 116 may be positioned or positionable in any pose, plane, and/or focal point. The pose includes a position and an orientation. As a result, an imaging device 112, surgical tool, or other object held by the robot 114 (or, more specifically, by the robotic arm 116) may be precisely positionable in one or more needed and specific positions and orientations.

The robotic arm(s) 116 may comprise one or more sensors that enable the processor 104 (or a processor of the robot 114) to determine a precise pose in space of the robotic arm (as well as any object or element held by or secured to the robotic arm).

In some embodiments, reference markers 202 (e.g., navigation markers) (shown in FIG. 2), may be placed on the robot 114 (including, e.g., on the robotic arm 116), the imaging device 112, an anatomical element such as an anatomical element 208 (shown in FIG. 2) or any other object in the surgical space. The reference markers 202 may be tracked by the navigation system 118, and the results of the tracking may be used by the robot 114 and/or by an operator of the system 100 or any component thereof. In some embodiments, the navigation system 118 can be used to track other components of the system (e.g., imaging device 112) and the system can operate without the use of the robot 114 (e.g., with the surgeon manually manipulating the imaging device 112 and/or one or more surgical tools, based on information and/or instructions generated by the navigation system 118, for example).

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers 202, navigated trackers, or other objects within the operating room or other room in which some or all of the system 100 is located. The one or more cameras may be optical cameras, infrared cameras, or other cameras. In some embodiments, the navigation system 118 may comprise one or more electromagnetic sensors. In various embodiments, the navigation system 118 may be used to track a position and orientation (e.g., a pose) of the imaging device 112, the robot 114 and/or robotic arm 116, and/or one or more surgical tools (or, more particularly, to track a pose of a navigated tracker attached, directly or indirectly, in fixed relation to the one or more of the foregoing). The navigation system 118 may include a display for displaying one or more images from an external source (e.g., the computing device 102, imaging device 112, or other source) or for displaying an image and/or video stream from the one or more cameras or other sensors of the navigation system 118. In some embodiments, the system 100 can operate without the use of the navigation system 118. The navigation system 118 may be configured to provide guidance to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

The database 130 may store information that correlates one coordinate system to another (e.g., one or more robotic coordinate systems to a patient coordinate system and/or to a navigation coordinate system). The database 130 may additionally or alternatively store, for example, one or more surgical plans (including, for example, pose information about a target and/or image information about a patient's anatomy at and/or proximate the surgical site, for use by the robot 114, the navigation system 118, and/or a user of the computing device 102 or of the system 100); one or more images useful in connection with a surgery to be completed by or with the assistance of one or more other components of the system 100; and/or any other useful information. The database 130 may be configured to provide any such information to the computing device 102 or to any other device of the system 100 or external to the system 100, whether directly or via the cloud 134. In some embodiments, the database 130 may be or comprise part of a hospital image storage system, such as a picture archiving and communication system (PACS), a health information system (HIS), and/or another system for collecting, storing, managing, and/or transmitting electronic medical records including image data.

The cloud 134 may be or represent the Internet or any other wide area network. The computing device 102 may be connected to the cloud 134 via the communication interface 108, using a wired connection, a wireless connection, or both. In some embodiments, the computing device 102 may communicate with the database 130 and/or an external device (e.g., a computing device) via the cloud 134.

The system 100 or similar systems may be used, for example, to carry out one or more aspects of any of the method 300 described herein. The system 100 or similar systems may also be used for other purposes.

Turning to FIG. 2, a schematic illustration of a top view of an example one or more surgical landmarks 200 is illustrated. The one or more surgical landmarks 200 may comprise a reference marker 202 disposed on an anatomical element 208, an implant 204 implanted on an anatomical element 208, an anatomical element 208, and/or a portion 210 of an anatomical element 208. Though the foregoing elements are illustrated together, it will be appreciated that the surgical landmark 200 may comprise the reference marker 202, the implant 204, the anatomical element 208, the portion 210 of the anatomical element 208, and/or any other landmark in any combination thereof. For example, the one or more surgical landmarks 200 may comprise the anatomical element 208.

The reference marker 202 may be the same as or similar to the reference markers 202 described above. In the illustrated embodiment, the anatomical element 208 comprises, for example, a vertebra. It will be appreciated that in other embodiments, the anatomical element 208 may comprise any anatomical element of a patient such as, for example, a bone, an organ, soft tissue, hard tissue, or the like. In the illustrated embodiment, the implant 204 comprises a screw, though it will be appreciated that in other embodiments, the implant 204 may be any implant such as, for example, a cage, a pin, a rod, or a stent. The portion 210 of the anatomical element 208, as shown, comprises a wing of a vertebra. It will be appreciated that in other embodiments, the portion 210 may be any portion or combination of portions of any anatomical element 208.

In some embodiments, the one or more surgical landmarks 200 may comprise the reference marker 202, the implant 204, the anatomical element 208, and/or the portion 210 of the anatomical element 208 in, for example, a surgical procedure in which the surgical site is “open.” In other words, the one or more surgical landmarks 200 may comprise one or more of the reference marker 202, the implant 204, the anatomical element 208, the portion 210 of the anatomical element 208, and/or any combination thereof as these landmarks may be visible during the surgical procedure due to the open nature of the surgical site. For example, during an open spinal surgery, one or more vertebrae may be openly visible, and thus, the one or more landmarks may comprise the one or more vertebrae. In some embodiments, where the surgical site is “closed” as for example, in minimally invasive surgeries or endoscopic surgeries, the one or more landmarks 200 may comprise the reference marker 202 and/or the implant 204. For example, in a minimally invasive surgical spinal procedure, the reference marker 202 and/or the implant 204 may be visible (and thus, define the one or more landmarks 200), whereas the anatomical elements 208 (e.g., vertebrae) may not be openly visible, but may be visible through the field of view provided by an endoscope.

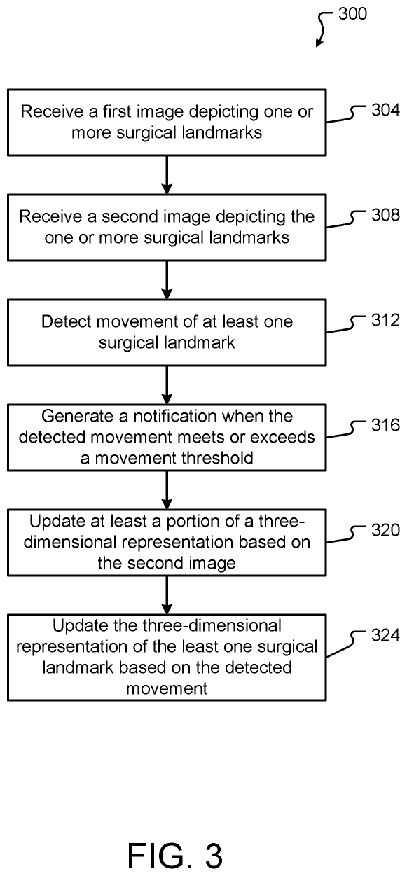

FIG. 3 depicts a method 300 that may be used, for example, for tracking one or more surgical landmarks.

The method 300 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 300. The at least one processor may perform the method 300 by executing elements stored in a memory such as the memory 106. The elements stored in memory and executed by the processor may cause the processor to execute one or more steps of a function as shown in method 300. One or more portions of a method 300 may be performed by the processor executing any of the contents of memory, such as an image processing 120, a segmentation 122, and/or a registration 124.

The method 300 comprises receiving a first image depicting one or more surgical landmarks (step 304). The one or more surgical landmarks may be the same as or similar to the one or more surgical landmarks 200. The first image may be, for example, a long film of a spine. The first image may be received via a user interface such as the user interface 110 and/or a communication interface such as the communication interface 108 of a computing device such as the computing device 102, and may be stored in a memory such as the memory 106 of the computing device. The first image may also be received from an external database or image repository (e.g., a hospital image storage system, such as a picture archiving and communication system (PACS), a health information system (HIS), and/or another system for collecting, storing, managing, and/or transmitting electronic medical records including image data), and/or via the Internet or another network. In other embodiments, the first image may be received or obtained from an imaging device such as the imaging device 112, which may be any imaging device such as an Mill scanner, a CT scanner, any other X-ray based imaging device, or an ultrasound imaging device. The first image may also be generated by and/or uploaded to any other component of a system such as the system 100. In some embodiments, the first image may be indirectly received via any other component of the system or a node of a network to which the system is connected.

The first image may be a two-dimensional image, a three-dimensional representation, or a set of two-dimensional images and/or three-dimensional representations. The first image may depict the one or more surgical landmarks, surgical instruments, surgical tools, or any component of a surgical operating setting. The one or more surgical landmarks may comprise a reference marker such as the reference marker 202 disposed on a corresponding anatomical element such as the anatomical element 208, an implant such as the implant 204 implanted on corresponding anatomical elements, one or more anatomical elements, and/or a portion such as the portion 210 of each of the one or more anatomical elements. In some embodiments, the first image may be captured preoperatively (e.g., before surgery) and may be stored in a system (e.g., a system 100) and/or one or more components thereof (e.g., a database 130). The stored first image may then be received (e.g., by a processor 104), as described above, preoperatively (e.g., before the surgery) and/or intraoperatively (e.g., during surgery). In some embodiments, the first image may depict multiple anatomical elements associated with the patient anatomy, including incidental anatomical elements (e.g., ribs or other anatomical objects on which a surgery or surgical procedure will not be performed) in addition to target anatomical elements (e.g., vertebrae or other anatomical objects on which a surgery or surgical procedure is to be performed). The first image may comprise various features corresponding to the patient's anatomy and/or anatomical elements (and/or portions thereof), including gradients corresponding to boundaries and/or contours of the various depicted anatomical elements, varying levels of intensity corresponding to varying surface textures of the various depicted anatomical elements, combinations thereof, and/or the like.

In embodiments where the one or more landmarks comprise a reference marker such as the reference marker 202, the first image may depict the reference marker. In some embodiments, the reference marker may have first markers visible in the first image and second markers visible to a navigation system such as the navigation system 118. For example, the reference marker may comprise metal balls that are detectable and/or trackable using an X-ray imaging device (including, for example, a C-arm, an O-arm, a fluoroscope), and infrared-reflecting spheres that are detectable and/or trackable by the navigation system. In some embodiments, the reference marker may be affixed to any portion of the patient including, for example, an anatomical element of the patient. It will be appreciated that the reference marker may have any number of markers visible and any marker may be visible in the first image and/or to a navigation system such as the navigation system 118.

Each first image may be processed using instructions or models stored in the memory that, when executed, enable image processing such as image processing 120 of the first image to identify the one or more landmarks in the first image. In other instances, each first image may be processed using instructions or models stored in the memory that, when executed, enable segmentation such as segmentation 122 of the first image to identify the one or more landmarks in the first image.

In some embodiments, the image processing and/or segmentation may use feature recognition to identify a feature of the one or more landmarks. In embodiments where the one or more landmarks comprises an anatomical element or a portion of an anatomical element a contour of a vertebrae, femur, or other bone, for example, may be identified in the first image. In other embodiments, the processor may use artificial intelligence or machine learning to identify the one or more landmarks. In such embodiments, a plurality of training first images may be provided to the processor, each training first image annotated to include identifying information about one or more landmarks in the first image. The processor, executed instructions stored in the memory or in another memory, may analyze the first images using a machine-learning algorithm and, based on the analysis, generate one or more models for identifying the one or more landmarks in a first image.

Each first image may also be registered using instructions or models stored in the memory that, when executed, enable registration such as the registration 124 of the first image to a patient. Registering the first image may correlate a coordinate space of the first image to a coordinate space of the patient.

The method 300 also comprises receiving a second image depicting the one or more surgical landmarks (step 308). Receiving the second image may be the same as or similar to the step 304 with respect to receiving an image. The second image may depict the same one or more surgical landmarks as depicted in the first image. In other embodiments, the second image may depict some of the one or more surgical landmarks depicted in the first image. In some embodiments, the second image may be processed using the image processing and/or segmentation to identify a shape, type, and/or morphology of the one or more landmark. In some embodiments, the one or more landmarks may be an implant and/or an anatomical element. It will be appreciated that in some embodiments, the one or more landmarks identified in the second image may facilitate automated anatomical landmark detection to match the identified landmarks from the second image to landmarks in the first image.

In some embodiments, the first image may be obtained preoperatively and the second image may be obtained intraoperatively. It will be appreciated that in other embodiments, the first image and the second image may be both obtained intraoperatively. In some embodiments, the second image may be received in real-time during a surgical procedure. In such embodiments, the step 308 may be continuously repeated and subsequent second image(s) may also be received in real-time. In other words, the imaging device may continuously obtain image(s) of the one or more landmarks throughout a surgical procedure. This enables tracking of the one or more landmarks, whether for unintended movement or confirmation of a planned movement.

In some embodiments, the imaging device comprises a first imaging device and a second imaging device. In such instances, the first imaging device may be used to obtain the first image at a first time, and the second imaging device may be used to obtain a second image at a second time after the first time. In other words, the second image may be received after the first image. In further instances, the first imaging device may obtain the first image using a first imaging modality and the second imaging device may obtain the second image using a second imaging modality. For example, the first imaging modality may use ionizing radiation (e.g., X-rays) and the second imaging modality may be free of ionizing radiation (e.g., ultrasound). It will be appreciated that the second imaging device may be, for example, any type of camera, an optical camera, a video camera, or any type of non-ionized medical imager such as, for example, an ultrasound device.

Embodiments provided herein also contemplate a scenario where the first and second images are captured at substantially the same time (e.g., synchronously), but from different imaging devices (e.g., from a first imaging device and a second imaging device, respectively). It should be appreciated that the images obtained at substantially the same time may be captured using different imaging modalities, may be taken from different perspective, and/or may have different fields of view. In some embodiments, the first image and second image may have an overlapping (partially or completely) field of view that includes the one or more surgical landmarks. In some embodiments, more than two images (e.g., a third image, a fourth image, . . . , a tenth image, etc.) may be captured and compared to any one of the first image, second image, or other subsequently-captured images.

The method 300 also comprises detecting movement of at least one surgical landmark (step 312). Detecting movement of the at least one surgical landmark may be based on a comparison of the first image (which may be received in step 304 above) with the second image (which may be received in step 308 above). More specifically, detecting the movement may comprise comparing a position of the at least one surgical landmark in the first image to the position of the at least one surgical landmark in the second image. In some embodiments, detecting movement of the at least one surgical landmark may comprise superimposing the second image over the first image and comparing differences between the surgical landmarks depicted in the first image and the second image. The differences may be determined by visually detecting the differences between the first image and the second image. In other instances, the differences may be determined automatically by, for example, a processor such as the processor 104. For example, the processor may compare each pixel of the first image to each corresponding pixel of the second image and differences in pixels may indicate a difference between the first image and the second image.

In embodiments where the first image and the second image are obtained intraoperatively, the movement or change detected may be relative to the patient's posture and orientation (e.g., prone, supine, etc.) as captured intraoperatively in the first image. In other words, in some embodiments, changes to the surgical landmarks (and thus, patient anatomy such as, for example, a spine) may correlate to surgically imparted changes and thus, the detected movement or changes may be compared directly to the first image.

In embodiments where the first image is taken preoperatively and the second image is taken intraoperatively, the movement or change detected to the surgical landmarks is determined as a function of both change in a patient's posture (e.g., a patient may be, for example, upright in the first image and supine in the second image) and surgically imparted changes. In other words, in some embodiments, changes to the surgical landmark may be due to both a difference in a patient's posture or orientation and a surgically imparted change.

In embodiments where the first image is taken preoperatively and the second image is taken intraoperatively and the first image and the second image are registered, then the second image may be updated using consecutive second images to determine how much change to a surgical landmark has been imparted to an anatomical element (such as, for example, a spine) intraoperatively. In other words, multiple second images may be obtained and subsequent second images may be compared to former second images to determine a change in the surgical landmarks (and thus, the anatomical element to which the surgical landmarks are attached to) between the former and subsequent second images. The detected movement or change can be compared to the first image, which may depict a pre-operative shape of the anatomical element. In such embodiments, the movement or changes detected can be used to assess, for example, a global alignment of a patient's spine and compensatory mechanisms.

The method 300 also comprises generating a notification when the detected movement meets or exceeds a movement threshold (step 316). The notification may be, for example, audible, visual, or a combination thereof. The notification may simply alert a user that movement has occurred. In other instances, the notification may specify landmark(s) that have moved.

The movement threshold may be based on an acceptable amount of movement that the at least one surgical landmark may exhibit. In some instances, no movement may be desired, however, a small movement threshold may be acceptable. In other instances, the at least one landmark may be expected to move due to, for example, a surgical step. As such, the movement threshold may be based on an acceptable difference between an actual movement and a predicted movement of the at least one surgical landmark. More specifically, a difference between an actual position of the at least one surgical landmark after the predicted movement and an expected position of the at least one surgical landmark may be determined to meet or exceed the movement threshold.

In some embodiments, the movement threshold may be received as input from, for example, a user such as a surgeon or other medical provider or may be provided in a surgical plan. In other embodiments, the movement threshold may be determined automatically by, for example, the processor preoperatively or intraoperatively. For example, the processor may execute a model which receives an expected position of one or more landmarks and/or a predicted movement of the one or more landmarks as input and out one or more movement thresholds. The model may be trained using, for example, historical movement thresholds, historical one or more landmarks, and/or one or more historical predicted movements of historical one or more landmarks.

In at least one embodiment where the movement threshold may be determined intraoperatively, the one or more landmarks may comprise one or more implants. In such embodiments, a trajectory of each implant may be obtained from, for example, a navigation system such as the navigation system 118. An expected position of each implant may be determined based on the determined trajectory, and the movement threshold may be based on the expected position.

The method 300 also comprises updating at least a portion of a three-dimensional representation based on the second image (step 320). In some embodiments, the first image may comprise one or more three-dimensional representations of the one or more surgical landmarks. In such embodiments, the second image may comprise one or more two-dimensional images of the one or more surgical landmarks. The second image may be received in, for example, step 308, though in other embodiments, the second image may be received in any manner.

In some embodiments, at least a portion of the three-dimensional representation may be updated. The updated portion may correspond to the portions depicted in the second image. In such instances, the second image may depict some of the one or more surgical landmarks and the surgical landmarks, which may correspond to the updated portion. Thus, the three-dimensional representation corresponding to the surgical landmarks depicted in the second image may be updated. For example, the three-dimensional representation may depict a first vertebra and a second vertebra and the second image may depict the first vertebra. In such examples, the three-dimensional representation of the first vertebra may be updated based on the second image.

In some embodiments, the entire three-dimensional representation may be updated. For example, the second image or a set of second images may depict the entire three-dimensional representation. In such embodiments, the second image or the set of second images may be used to update each landmark depicted in the three-dimensional representation.

Updating the three-dimensional representation may comprise updating a position of the landmark(s) based on the position of the landmark(s) as depicted in the second image. Updating the three-dimensional representation may also comprise updating a boundary such as, for example, a surface mesh of the landmark(s) based on the second image.

In some instances, the step 320 may not occur if movement is not detected in, for example, step 316. In other instances, the step 320 may occur regardless of detected movement. In still other embodiments, the step 320 may occur if a movement threshold, such as the movement threshold described with respect to step 216 is met or exceeded by the detected movement.

The method 300 also comprises updating the three-dimensional representation of the at least one surgical landmark based on the detected movement (step 324). The movement may be detected in, for example, step 312, though in other embodiments, the movement may be detected in any manner. The step 324 may be the same as or similar to the step 320, except that the three-dimensional representation is updated based on the detected movement of the at least one surgical landmark. For example, a new position of the at least one surgical landmark may be determined based on the detected movement or the new position may be received from a navigation system such as the navigation system 118. In such examples, a position of the at least one surgical landmark as depicted in the three-dimensional representation may be updated based on the new position.

In some instances, the step 324 may not occur if movement is not detected in, for example, step 316. In other instances, the step 324 may occur if a movement threshold, such as the movement threshold described with respect to step 216 is met or exceeded by the detected movement.

It will be appreciated that the step 308, the step 312, the step 316, the step 320, and/or the step 324 may be repeated throughout a surgical procedure. For example, subsequent second images may be received continuously in real-time and/or received at a time interval. In another example, movement may be continuously monitored throughout a surgical procedure, which may then cause a three-dimensional representation (e.g., the first image) to be updated. By repeating the step 308, the step 312, the step 316, the step 320, and/or the step 324, up-to-date positional information of one or more anatomical elements may be provided to a user such as a surgeon or other medical provider. In some embodiments, images from the step 308 may be used to continuously report a change in a position of the surgical landmark (and thus, the anatomical element to which the surgical landmark is attached to) as compared to an initial image (e.g., the first image). In such embodiments, the initial image may be updated to reflect the movement of the surgical landmark and/or the anatomical element to which the surgical landmark is attached to. As such, the surgeon may be able to determine if the anatomical elements are positioned correctly after a series of surgical moves. For example, during a spinal surgery, a surgeon may track actual movement and an actual alignment of a patient's spine and compare it to an expected movement and/or expected alignment (as may be stored, for example, in a surgical plan or obtained from, for example, a predictive model that predicts a surgical outcome). In another example, the surgeon may track the movement and alignment of a patient's spine and determine whether the changes in the alignment and repositioning are adequate or whether further intervention is desired. In such embodiments, the surgeon may also take into account other relevant factors such as, for example, a patient's age, a patient's history, etc. Thus, the systems, methods, and devices provided beneficially provide up-to-date positional information of anatomical elements during a surgical operation.

The present disclosure encompasses embodiments of the method 300 that comprise more or fewer steps than those described above, and/or one or more steps that are different than the steps described above.

As noted above, the present disclosure encompasses methods with fewer than all of the steps identified in FIG. 3 (and the corresponding description of the method 300), as well as methods that include additional steps beyond those identified in FIG. 3 (and the corresponding description of the method 300). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A system for tracking one or more surgical landmarks comprising:
an imaging device;
a processor; and
a memory storing data for processing by the processor, the data, when processed, causing the processor to:
receive a first image depicting one or more surgical landmarks;
receive a second image from the imaging device depicting the one or more surgical landmarks, the second image received after the first image;
detect movement of at least one surgical landmark of the one or more surgical landmarks based on a comparison of the first image with the second image; and
generate a notification for an operating room personnel when the detected movement meets or exceeds a movement threshold.

2. The system of claim 1, wherein the imaging device obtains images of a patient, including the second image, free of ionizing radiation.

3. The system of claim 1, wherein the one or more surgical landmarks comprise a reference marker disposed on corresponding anatomical elements, one or more implants implanted on corresponding anatomical elements, one or more anatomical elements, and/or a portion of each of one or more anatomical elements.

4. The system of claim 1, wherein detecting the movement of the at least one surgical landmark comprises comparing a position of the at least one surgical landmark in the first image to the position of the at least one surgical landmark in the second image.

5. The system of claim 2, wherein the first image is obtained preoperatively and the second image is obtained intraoperatively.

6. The system of claim 1, wherein the imaging device is a second imaging device and the system further comprises a first imaging device, wherein the first image is obtained from the first imaging device and the second image is obtained from the second imaging device.

7. The system of claim 6, wherein the first imaging device uses a first imaging modality and the second imaging device uses a second imaging modality different from the first imaging modality.

8. The system of claim 6, wherein the first image comprises a three-dimensional representation of the one or more surgical landmarks, wherein the first imaging device obtains images using ionizing radiation.

9. The system of claim 8, wherein the memory stores further data for processing by the processor that, when processed, causes the processor to:
update at least a portion of the three-dimensional representation based on the second image.

10. The system of claim 9, wherein the second image comprises a two-dimensional representation of the one or more surgical landmarks.

11. The system of claim 8, wherein the memory stores further data for processing by the processor that, when processed, causes the processor to:
update the three-dimensional representation of the at least one surgical landmark based on the detected movement of the at least one surgical landmark.

12. The system of claim 1, wherein the threshold is based on a predicted position of the at least one surgical landmark after a surgical step is performed.

13. The system of claim 1, wherein the second image is received in real-time.

14. A device for tracking an anatomical element comprising:
a processor; and
a memory storing data for processing by the processor, the data, when processed, causing the processor to:
receive a first image depicting one or more surgical landmarks;
receive a second image depicting the one or more surgical landmarks, the second image received after the first image;
detect movement of at least one surgical landmark of the one or more surgical landmarks based on the first image and the second image; and
update the first image based on the detected movement.

15. The device of claim 14, wherein detecting the movement of the at least one surgical landmark comprises comparing a position of the at least one surgical landmark in the first image with a position of the at least one surgical landmark in the second image.

16. The device of claim 14, wherein the one or more surgical landmarks comprise a reference marker disposed on corresponding anatomical elements, one or more implants implanted on corresponding anatomical elements, one or more anatomical elements, and/or a portion of each of one or more anatomical elements.

17. The device of claim 14, wherein the first image is at least one of a two-dimensional or three-dimensional representation of the one or more surgical landmarks and the second image is at least one of a two-dimensional or three-dimensional representation of the one or more surgical landmarks, wherein the first image is obtained from a first imaging device using a first imaging modality and the second image is obtained from a second imaging device using a second imaging modality.

18. The device of claim 17, wherein the first imaging modality uses ionizing radiation and the second imaging modality is free of ionizing radiation.

19. The device of claim 14, wherein updating the first image occurs when the detected movement meets or exceeds a movement threshold.

20. A system for tracking one or more surgical landmarks comprising:
a first imaging device using a first imaging modality;
a second imaging device using a second imaging modality;
a processor;
a memory storing data for processing by the processor, the data, when processed, causing the processor to:
receive a first image from the first imaging device, the first image depicting one or more surgical landmarks;
receive a second image from the second imaging device, the second image depicting the one or more surgical landmarks;
detect movement of at least one surgical landmark of the one or more surgical landmarks based on the first image and the second image; and
generate a notification when the detected movement meets a threshold.
